(19) United States
(12) Patent Application Publication
(10) Pub. No.: US 2025/0315948 A1
(43) Pub. Date: Oct. 9, 2025

(54) ULTRASOUND IMAGE ANALYSIS APPARATUS AND ULTRASOUND IMAGE ANALYSIS PROGRAM

(71) Applicant: FUJIFILM Corporation, Tokyo (JP)

(72) Inventor: Yushi TSUBOTA, Tokyo (JP)

(73) Assignee: FUJIFILM Corporation, Tokyo (JP)

(21) Appl. No.: 19/170,050

(22) Filed: Apr. 3, 2025

(30) Foreign Application Priority Data
Apr. 8, 2024 (JP) 2024-062206

(51) Int. Cl. G06T 7/00; A61B 8/08; G06T 7/11; G06T 7/136

(52) U.S. Cl. CPC G06T 7/0012; A61B 8/0825; G06T 7/11; G06T 7/136; G06T 2207/10132; G06T 2207/20104; G06T 2207/30068

(57) ABSTRACT

A mammary gland region specifying unit specifies a mammary gland region in a target breast ultrasound image formed by transmitting and receiving an ultrasound wave to and from a breast. A fat region specifying unit specifies a fat region in the target breast ultrasound image. A threshold value specifying unit specifies a brightness threshold value based on a brightness value of each pixel constituting the fat region. A GTC calculation unit divides a pixel group constituting the mammary gland region into a high-brightness pixel of which a brightness value is equal to or more than a brightness threshold value and a low-brightness pixel of which a brightness value is less than the brightness threshold value. The GTC calculation unit calculates a GTC that is a parameter indicating a ratio of the number of low-brightness pixels in the mammary gland region.

### CROSS REFERENCE TO RELATED APPLICATION

This application claims priority to Japanese Patent Application No. 2024-062206 filed on Apr. 8, 2024, which is incorporated herein by reference in their entireties including the specifications, claims, drawings, and abstracts.

### BACKGROUND OF THE INVENTION

### 1. Field of the Invention

The present specification discloses improvements in an ultrasound image analysis apparatus and an ultrasound image analysis program.

### 2. Description of the Related Art

In the related art, it is known that a mammary gland density in a breast is a risk factor for a breast cancer. The mammary gland density can be measured by a mammography apparatus. Meanwhile, in the related art, from the viewpoint of reducing pain of a subject or the like, a technique of evaluating a risk of the breast cancer using a breast ultrasound image formed by transmitting and receiving an ultrasound wave to and from the breast has been proposed.

In detail, it has been proposed to evaluate the risk of the breast cancer by calculating a parameter called a glandular tissue component (GTC) that is considered to have a high correlation with the mammary gland density using the breast ultrasound image. Specifically, a mammary gland region (a region between a subcutaneous fat region and a retromammary fat region in the vicinity of a pectoralis major muscle in a depth direction from a body surface) is specified in the breast ultrasound image, and the GTC is calculated based on an area ratio of an equal echo region (a region formed of a milk duct and a lobule and a surrounding stroma and having a low-brightness stroma region with respect to a high-brightness edematous stroma region) in the mammary gland region. The larger GTC indicates the higher mammary gland density, that is, the higher risk of the breast cancer.

For example, JP2023-077810A and JP2023-077820A disclose an ultrasound image analysis apparatus that specifies a breast region, which is a region between a skin and a pectoralis major muscle, in a breast ultrasound image, specifies a mammary gland region, which is a region between a front boundary line and a rear boundary line in the breast region, binarizes the mammary gland region with a predetermined brightness threshold value, sets a high-brightness region as an edematous stroma region and a low-brightness region as a GTC region (equal echo region), and calculates a ratio of the GTC region to the mammary gland region.

### SUMMARY OF THE INVENTION

Meanwhile, in order to calculate the GTC by using the breast ultrasound image, it is necessary to divide each pixel of the mammary gland region into a high-brightness pixel (corresponding to the edematous stroma region) and a low-brightness pixel (corresponding to the equal echo region) based on a brightness value thereof. The classification into the high-brightness pixel and the low-brightness pixel is performed by using the brightness threshold value. The brightness threshold value is an important parameter that directly affects a value of the GTC.

An ultrasound diagnostic apparatus or an ultrasound image processing apparatus (for example, a computer such as a server) generally has a function of adjusting a brightness value of each pixel constituting a breast ultrasound image (hereinafter, may be simply referred to as a brightness value of the breast ultrasound image). Therefore, the brightness value of the breast ultrasound image can be set to various values by a doctor, a facility (for example, a hospital), or the like. For example, a certain doctor may set the brightness value of the breast ultrasound image to be high overall, another doctor may set the brightness value of the breast ultrasound image to be low overall, and the like.

In a case where the brightness value of the breast ultrasound image can be adjusted as a whole, in a case where the brightness threshold value used for the GTC calculation is set to a predetermined value, the GTC can be changed according to the brightness value of the breast ultrasound image adjusted as a whole. As a result, an appropriate GTC may not be calculated.

An object of an ultrasound image analysis apparatus disclosed in the present specification is to appropriately calculate a GTC by using a breast ultrasound image formed by transmitting and receiving an ultrasound wave to and from a breast.

According to the present specification, there is provided an ultrasound image analysis apparatus comprising: a mammary gland region specifying unit that specifies a mammary gland region in a target breast ultrasound image, which is a processing target, formed by transmitting and receiving an ultrasound wave to and from a breast; a fat region specifying unit that specifies a fat region in a breast ultrasound image formed by transmitting and receiving an ultrasound wave to and from a breast; a threshold value specifying unit that specifies a brightness threshold value based on a brightness value of each pixel constituting the fat region; a GTC calculation unit that divides a pixel group constituting the mammary gland region into a high-brightness pixel having a brightness value equal to or more than the brightness threshold value and a low-brightness pixel having a brightness value less than the brightness threshold value, and calculates a GTC indicating a ratio of the number of low-brightness pixels in the mammary gland region; and a provision processing unit that provides the GTC to a user.

The target breast ultrasound image and the specified mammary gland region may be presented to the user, the mammary gland region specifying unit may correct the mammary gland region in response to an instruction from the user, and the GTC calculation unit may calculate a GTC indicating a ratio of the number of low-brightness pixels in the mammary gland region after the correction.

The breast ultrasound image may be the target breast ultrasound image, the ultrasound image analysis apparatus may further comprise a brightness value adjustment unit that adjusts a brightness value of each pixel constituting the target breast ultrasound image, the threshold value specifying unit may specify the brightness threshold value based on a brightness value after the adjustment of each pixel constituting the fat region of the target breast ultrasound image, and the GTC calculation unit may calculate a GTC indicating a ratio of the number of low-brightness pixels in the mammary gland region of the target breast ultrasound image of which the brightness value is adjusted.

The target breast ultrasound image and the specified fat region may be presented to the user, the fat region specifying unit may correct the fat region in response to an instruction from the user, and the threshold value specifying unit may specify the brightness threshold value based on a brightness value of each pixel constituting the fat region after the correction.

The provision processing unit may notify the user in a case where a variation in the brightness value of each pixel constituting the fat region is equal to or more than a variation threshold value.

The threshold value specifying unit may specify a maximum brightness threshold value in a brightness threshold value width that is an acceptable width of the brightness threshold value and a minimum brightness threshold value in the brightness threshold value width, based on a variation in the brightness value of each pixel constituting the fat region, the GTC calculation unit may calculate a maximum GTC that is the GTC based on the maximum brightness threshold value and a minimum GTC that is the GTC based on the minimum brightness threshold value, and the provision processing unit may provide at least one of the maximum GTC and the minimum GTC or a difference between the maximum GTC and the minimum GTC to the user, or may notify the user in a case where the difference between the maximum GTC and the minimum GTC is equal to or more than a GTC threshold value.

In addition, according to the present specification, there is provided an ultrasound image analysis program causing a computer to function as: a mammary gland region specifying unit that specifies a mammary gland region in a target breast ultrasound image, which is a processing target, formed by transmitting and receiving an ultrasound wave to and from a breast; a fat region specifying unit that specifies a fat region in a breast ultrasound image formed by transmitting and receiving an ultrasound wave to and from a breast; a threshold value specifying unit that specifies a brightness threshold value based on a brightness value of each pixel constituting the fat region; a GTC calculation unit that divides a pixel group constituting the mammary gland region into a high-brightness pixel having a brightness value equal to or more than the brightness threshold value and a low-brightness pixel having a brightness value less than the brightness threshold value, and calculates a GTC indicating a ratio of the number of low-brightness pixels in the mammary gland region; and a provision processing unit that provides the GTC to a user.

With the ultrasound image analysis apparatus disclosed in the present specification, a GTC can be appropriately calculated by using a breast ultrasound image formed by transmitting and receiving an ultrasound wave to and from a breast.

### BRIEF DESCRIPTION OF THE DRAWINGS

FIG. 1 is a schematic configuration diagram of an ultrasound diagnostic apparatus according to the present embodiment.

FIG. 2 is a diagram illustrating an example of a target breast ultrasound image and a mammary gland region.

FIG. 3 is a diagram illustrating an example of a correction screen of the mammary gland region.

FIG. 4 is a diagram illustrating an example of a fat region in the target breast ultrasound image.

FIG. 5 is a diagram illustrating an example of a correction screen of a fat region.

FIG. 6 is a conceptual diagram illustrating a high-brightness pixel and a low-brightness pixel in a correction mammary gland region.

FIG. 7 is a diagram illustrating an example of a GTC display screen.

FIG. 8 is a diagram illustrating another example of the GTC display screen.

FIG. 9 is a flowchart illustrating a flow of a process of the ultrasound diagnostic apparatus according to the present embodiment.

### DESCRIPTION OF THE PREFERRED EMBODIMENTS

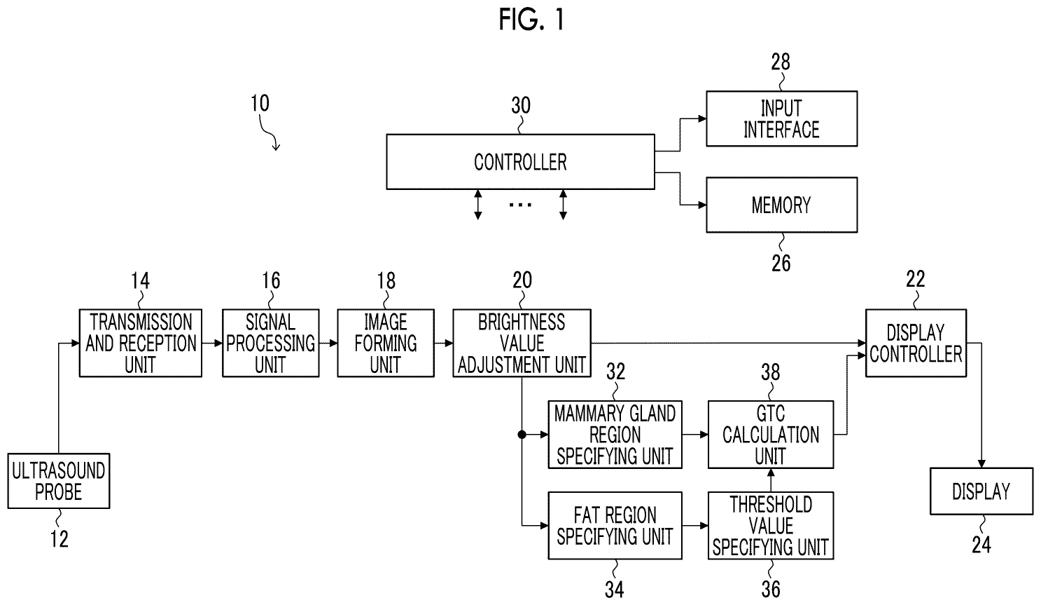

FIG. 1 is a schematic configuration diagram of a configuration of an ultrasound diagnostic apparatus 10 as an ultrasound image analysis apparatus according to the present embodiment. The ultrasound diagnostic apparatus 10 is a medical apparatus that is installed in a medical institution such as a hospital.

The ultrasound diagnostic apparatus 10 is an apparatus that scans a subject with an ultrasound beam to generate an ultrasound image as a medical image based on a reception signal obtained by the scanning. In particular, in the present embodiment, the ultrasound diagnostic apparatus 10 forms a breast ultrasound image, based on a reception signal obtained by transmitting and receiving an ultrasound wave to and from a breast of the subject.

A transmission and reception unit 14, a signal processing unit 16, an image forming unit 18, a brightness value adjustment unit 20, a display controller 22, a mammary gland region specifying unit 32, a fat region specifying unit 34, a threshold value specifying unit 36, and a GTC calculation unit 38 included in the ultrasound diagnostic apparatus 10 are configured with a processor. The processor is configured to include at least one of a general-purpose processing apparatus (for example, a central processing unit (CPU) or the like) or a dedicated processing apparatus (for example, a graphics processing unit (GPU), an application specific integrated circuit (ASIC), a field programmable gate array (FPGA), a programmable logic device, or the like). The processor may be configured with cooperation between a plurality of processing apparatuses existing at physically separated positions, instead of being configured with one processing apparatus. In addition, each of the units described above may be implemented by cooperation between hardware, such as a processor, and software.

An ultrasound probe 12 is a device that transmits and receives an ultrasound wave to and from the subject. The ultrasound probe 12 has a transducer element array including a plurality of transducer elements that transmit and receive the ultrasound wave to and from the subject. In particular, in the present embodiment, the ultrasound probe 12 is in contact with the breast of the subject, and transmits and receives the ultrasound wave to and from the breast.

The transmission and reception unit 14 transmits a transmission signal to the ultrasound probe 12 (specifically, each transducer element of the transducer element array) under control of a controller 30 (to be described below). As a result, the ultrasound wave is transmitted from each transducer element toward the breast of the subject.

In addition, the transmission and reception unit 14 receives a reception signal from each transducer element that has received a reflected wave from the breast of the subject. The transmission and reception unit 14 includes an adder and a plurality of delayers corresponding to each transducer element, and a phase matching and addition process of matching and adding phases of the reception signal from each transducer element is performed by the adder and the plurality of delayers. As a result, a reception beam signal is formed in which information indicating a signal intensity of the reflected wave from the subject is arranged in a depth direction of the subject.

The signal processing unit 16 executes various types of signal processing including a filtering process of applying a bandpass filter, a detection process, and the like on the reception beam signal from the transmission and reception unit 14.

The image forming unit 18 forms an ultrasound tomographic image (B-mode image) based on the reception beam signal on which the signal processing unit 16 performs the signal processing. In particular, in the present embodiment, the image forming unit 18 forms a breast ultrasound image which is a B-mode image. In the present specification, a breast ultrasound image as a processing target, which is used in a GTC calculation, is referred to as a target breast ultrasound image. In a case of simply describing the breast ultrasound image, the breast ultrasound image means not only the target breast ultrasound image but also other breast ultrasound images. There may be a plurality of target breast ultrasound images. The plurality of target breast ultrasound images may have cross sections which are different from each other. In a case where there are a plurality of target breast ultrasound images, each unit to be described below performs a process on each target breast ultrasound image.

The brightness value adjustment unit 20 adjusts a brightness value of each pixel constituting the breast ultrasound image formed by the image forming unit 18. The brightness value adjustment unit 20 may automatically adjust the brightness value of the breast ultrasound image, based on a preset value set in advance in the ultrasound diagnostic apparatus 10. In addition, the brightness value adjustment unit 20 may adjust the brightness value of the breast ultrasound image in response to an instruction from a user such as a doctor. In that case, the user may be able to designate the adjustment amount of brightness value for each depth.

The display controller 22 performs control to display the target breast ultrasound tomographic image formed by the image forming unit 18 on a display 24. In addition, the display controller 22 displays various types of information such as a calculation result of the GTC calculation unit 38, which will be described below, on the display 24. Details of the information displayed on the display 24 by the display controller 22 will be described below.

The display 24 is a display device configured with, for example, a liquid crystal display, an organic electro luminescence (EL), or the like.

A memory 26 is configured to include a hard disk drive (HDD), a solid state drive (SSD), an embedded multi media card (eMMC), a read only memory (ROM), a random access memory (RAM), or the like. An ultrasound image analysis program is stored in the memory 26 to operate each unit of the ultrasound diagnostic apparatus 10. The ultrasound image analysis program can also be stored in, for example, a computer readable non-transitory storage medium such as a universal serial bus (USB) memory or a CD-ROM. The ultrasound diagnostic apparatus 10 can read and execute the ultrasound image analysis program from such a storage medium.

An input interface 28 is configured with, for example, a button, a trackball, a touch panel, and the like. The input interface 28 is used to input a command from a user to the ultrasound diagnostic apparatus 10.

The controller 30 is configured to include at least one of a general-purpose processor (for example, a CPU or the like) or a dedicated processor (for example, a GPU, an ASIC, an FPGA, or a programmable logic device). The controller 30 may be configured with cooperation of a plurality of processing apparatuses existing at physically separated positions, instead of being configured with one processing apparatus. The controller 30 controls each unit of the ultrasound diagnostic apparatus 10 according to the ultrasound image analysis program stored in the memory 26.

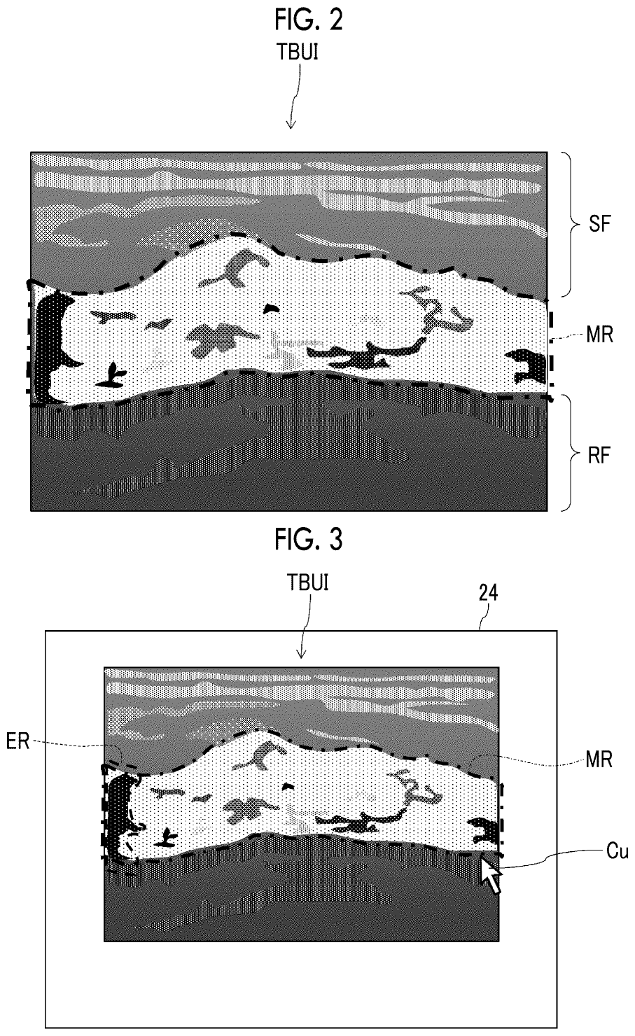

The mammary gland region specifying unit 32 specifies a mammary gland region in the target breast ultrasound image. FIG. 2 is a diagram illustrating an example of a target breast ultrasound image TBUI and a mammary gland region MR. The mammary gland region MR is a region between a subcutaneous fat region SF located immediately below a body surface and a retromammary fat region RF in the vicinity of a pectoralis major muscle, in the depth direction. In FIG. 2, the mammary gland region MR is indicated by a one-dot chain line.

The mammary gland region specifying unit 32 can specify the mammary gland region MR by analyzing the target breast ultrasound image TBUI. For example, by using a breast ultrasound image and the mammary gland region MR which is manually designated in the breast ultrasound image as learning data, the mammary gland region specifying unit 32 can specify the mammary gland region MR in the target breast ultrasound image TBUI by inputting the target breast ultrasound image TBUI to a learning model (for example, a convolutional neural network) trained to specify the mammary gland region MR from the breast ultrasound image.

In addition, the mammary gland region specifying unit 32 may specify the mammary gland region MR in the target breast ultrasound image TBUI in response to an instruction from a user. For example, the display controller 22 may display the target breast ultrasound image TBUI on the display 24, the user may input an instruction indicating the mammary gland region MR to the ultrasound diagnostic apparatus 10 by using the input interface 28, and the mammary gland region specifying unit 32 may specify the mammary gland region MR based on the input instruction.

The display controller 22 may display the target breast ultrasound image TBUI and the mammary gland region MR specified by the mammary gland region specifying unit 32 on the display 24 to present the target breast ultrasound image TBUI and the mammary gland region MR to the user. The user may be able to correct the mammary gland region MR on the screen (correction screen for the mammary gland region MR).

FIG. 3 is a diagram illustrating an example of a correction screen of the mammary gland region MR. In the correction screen illustrated in FIG. 3, a user can input a correction instruction for the mammary gland region MR to the ultrasound diagnostic apparatus 10, by using a cursor Cu or the like displayed on the screen. The mammary gland region specifying unit 32 corrects the mammary gland region MR in response to the correction instruction from the user. The user may be able to designate a removal region ER as one of the correction instructions for the mammary gland region MR. The removal region ER is a region to be excluded from the mammary gland region MR. In FIG. 3, the removal region ER is indicated by a broken line. The mammary gland region specifying unit 32 sets a portion obtained by excluding the removal region ER from the mammary gland region MR as the mammary gland region MR after correction.

In the correction screen, the display controller 22 may display the mammary gland region MR and the removal region ER in different modes. As a result, the user can easily discriminate the mammary gland region MR and the removal region ER. For example, a boundary line of the mammary gland region MR may be displayed in blue, and a boundary line of the removal region ER may be displayed in red. In addition, the entire mammary gland region MR may be displayed in blue, and the entire removal region ER may be displayed in red. In this case, the mammary gland region MR and the removal region ER may be colored in a translucent manner such that visibility of the target breast ultrasound image TBUI itself is not reduced. Alternatively, as illustrated in FIG. 3, line types of the boundary line of the mammary gland region MR and the boundary line of the removal region ER may be different from each other.

The fat region specifying unit 34 specifies a fat region in a breast ultrasound image. In the present embodiment, the fat region specifying unit 34 specifies the fat region in the target breast ultrasound image. Meanwhile, as will be described below, the fat region specifying unit 34 may specify the fat region in a breast ultrasound image other than the target breast ultrasound image under a certain condition.

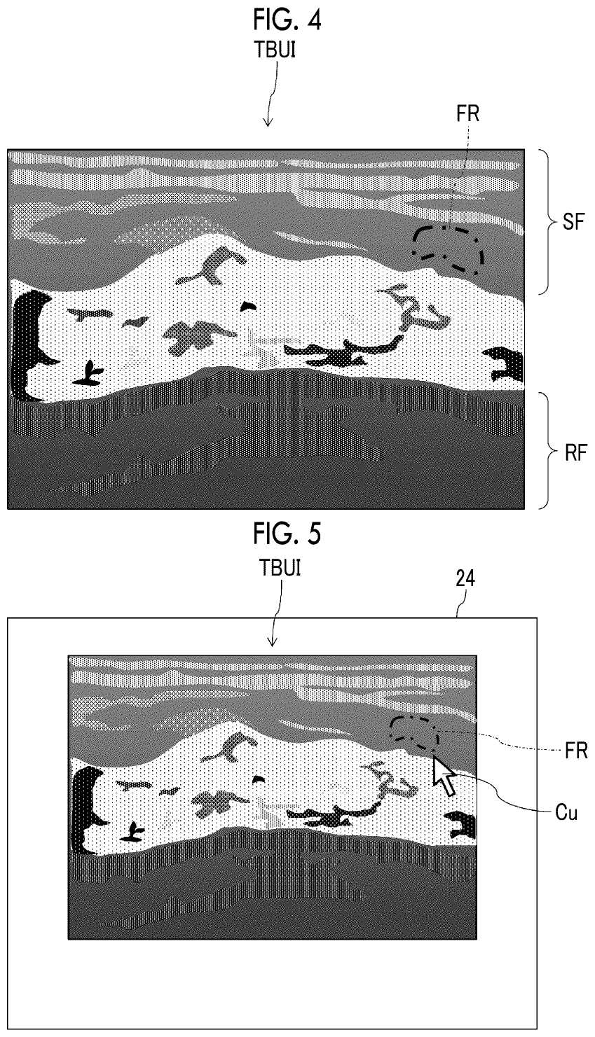

FIG. 4 is a diagram illustrating an example of a fat region FR in the target breast ultrasound image TBUI. The fat region FR is a region in the subcutaneous fat region SF or the retromammary fat region RF. In FIG. 4, the fat region FR is indicated by a one-dot chain line.

The fat region specifying unit 34 can specify the fat region FR by analyzing the target breast ultrasound image TBUI. For example, by using a breast ultrasound image and the fat region FR which is manually designated in the breast ultrasound image as learning data, the fat region specifying unit 34 can specify the fat region FR in the target breast ultrasound image TBUI by inputting the target breast ultrasound image TBUI to a learning model (for example, a convolutional neural network) trained to specify the fat region FR from the breast ultrasound image.

In addition, the fat region specifying unit 34 may specify the fat region FR in the target breast ultrasound image TBUI in response to an instruction from a user. For example, the display controller 22 may display the target breast ultrasound image TBUI on the display 24, the user may input an instruction indicating the fat region FR to the ultrasound diagnostic apparatus 10 by using the input interface 28, and the fat region specifying unit 34 may specify the fat region FR based on the input instruction.

The display controller 22 may display the target breast ultrasound image TBUI and the fat region FR specified by the fat region specifying unit 34 on the display 24 to present the target breast ultrasound image TBUI and the fat region FR to the user. The user may be able to correct the mammary gland region MR on the screen (fat region FR correction screen).

FIG. 5 is a diagram illustrating an example of a correction screen of the mammary gland region MR. In the correction screen illustrated in FIG. 5, a user can input a correction instruction for the fat region FR to the ultrasound diagnostic apparatus 10, by using the cursor Cu or the like displayed on the screen. The fat region specifying unit 34 corrects the fat region FR in response to the correction instruction from the user.

As will be described below, the fat region FR is used to specify a brightness threshold value for dividing a pixel group constituting the mammary gland region MR into a high-brightness pixel and a low-brightness pixel. Specifically, the brightness threshold value is specified based on a brightness value of each pixel constituting the fat region FR. Therefore, the fat region specifying unit 34 may specify the fat region FR in a breast ultrasound image other than the target breast ultrasound image TBUI as long as the fat region FR has the same brightness value as the fat region FR of the target breast ultrasound image TBUI. For example, a plurality of breast ultrasound images acquired by the same doctor may be often adjusted such that brightness values are similar to each other. Therefore, the fat region specifying unit 34 may specify the fat region FR in another breast ultrasound image (not necessarily the same subject) acquired by the same doctor as the target breast ultrasound image TBUI.

The threshold value specifying unit 36 specifies the brightness threshold value based on the brightness value of each pixel constituting the fat region FR specified by the fat region specifying unit 34. For example, the threshold value specifying unit 36 sets a representative value (for example, an average value) of a plurality of brightness values of a plurality of pixels constituting the fat region FR as the brightness threshold value. Alternatively, the threshold value specifying unit 36 may add or subtract a predetermined bias value to the representative value of the plurality of brightness values of the plurality of pixels constituting the fat region FR, and use the added or subtracted value as the brightness threshold value.

In a case where the fat region FR is corrected by the fat region specifying unit 34, the threshold value specifying unit 36 may specify the brightness threshold value based on a brightness value of each pixel constituting the fat region FR after the correction.

In addition, in a case where a brightness value of the target breast ultrasound image TBUI is adjusted by the brightness value adjustment unit 20, the threshold value specifying unit 36 may specify a brightness threshold value based on the brightness value after the adjustment of each pixel constituting the fat region FR of the target breast ultrasound image TBUI.

The threshold value specifying unit 36 may calculate a variation in brightness value of each pixel constituting the fat region FR (fat region FR after the correction in a case where the fat region FR is corrected). In a case where the variation in brightness value of each pixel constituting the fat region FR is large, the brightness threshold value may not be appropriate. Therefore, the display controller 22 may notify the user in a case where the variation in brightness value of each pixel constituting the fat region FR is equal to or more than a predetermined variation threshold value. With the notification, the user can correct or re-select the fat region FR.

In the present embodiment, the threshold value specifying unit 36 calculates a standard deviation s of the brightness value of each pixel constituting the fat region FR, as a parameter indicating a variation in brightness value of each pixel constituting the fat region FR. The display controller 22 notifies the user in a case where the standard deviation s of the brightness value of each pixel constituting the fat region FR is equal to or more than a predetermined standard deviation threshold value.

Further, the threshold value specifying unit 36 may specify a maximum brightness threshold value in a brightness threshold value width that is an acceptable width of the brightness threshold value and a minimum brightness threshold value in the brightness threshold value width, based on the variation in brightness value of each pixel constituting the fat region FR. In the present embodiment, the threshold value specifying unit 36 sets m+s obtained by adding the standard deviation s to a representative value (here, an average value m) of a plurality of brightness values of a plurality of pixels constituting the fat region FR as the maximum brightness threshold value, and sets m-s obtained by subtracting the standard deviation s from the average value m as the minimum brightness threshold value. A method of using the maximum brightness threshold value and the minimum brightness threshold value will be described below. In the present specification, a brightness threshold value based on the representative value of the plurality of brightness values of the plurality of pixels constituting the fat region FR may be referred to as a standard brightness threshold value to distinguish the brightness threshold value from the maximum brightness threshold value and the minimum brightness threshold value. In a case of being simply described as a brightness threshold value, it means the standard brightness threshold value.

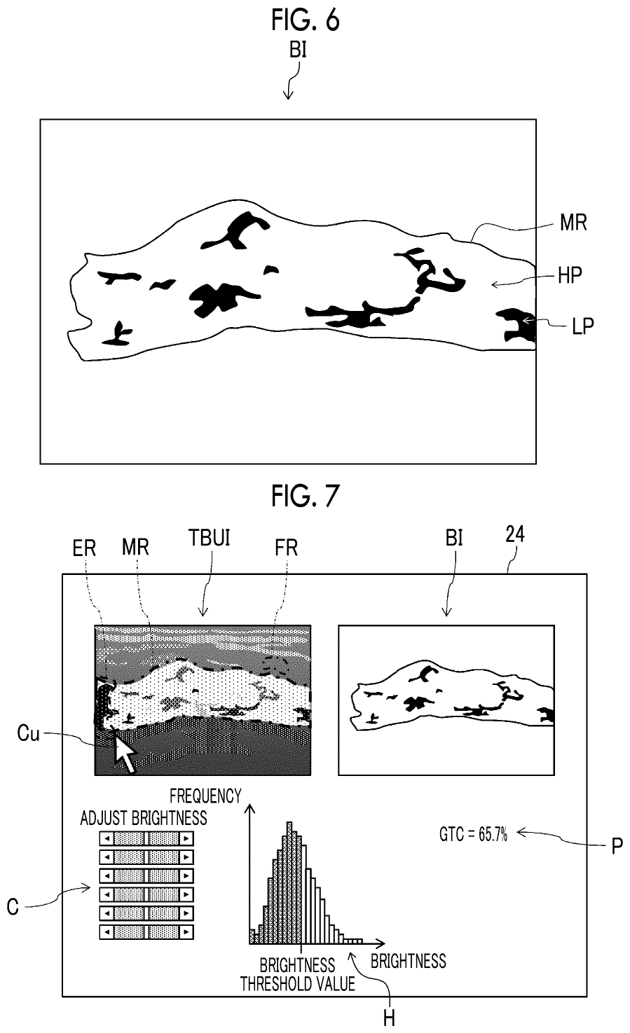

The GTC calculation unit 38 calculates a GTC based on the target breast ultrasound image TBUI. Specifically, first, the GTC calculation unit 38 divides a pixel group constituting the mammary gland region MR specified by the mammary gland region specifying unit 32 into a high-brightness pixel of which a brightness value is equal to or more than a brightness threshold value specified by the threshold value specifying unit 36 and a low-brightness pixel in which the brightness value is less than the brightness threshold value. In the present embodiment, as illustrated in FIG. 6, the GTC calculation unit 38 forms a binarized image BI in which a brightness value of a high-brightness pixel HP is set to the maximum brightness and a brightness value of a low-brightness pixel LP is set to the minimum brightness in the mammary gland region MR. The GTC calculation unit 38 calculates a GTC that is a parameter indicating a ratio of the number of low-brightness pixels LP in the mammary gland region MR.

In the present embodiment, the GTC calculation unit 38 calculates (the number of low-brightness pixels LP/the number of pixels of the mammary gland region MR)×100 as the GTC.

Meanwhile, the GTC may be calculated by another method as long as the ratio of the number of low-brightness pixels LP in the mammary gland region MR is indicated. For example, since a total of the number of high-brightness pixels HP and the number of low-brightness pixels LP is the number of pixels of the mammary gland region MR, a ratio of the number of high-brightness pixels HP to the number of low-brightness pixels LP may be calculated as the GTC.

In a case where the mammary gland region MR is corrected by the mammary gland region specifying unit 32, the GTC calculation unit 38 may calculate a GTC indicating the ratio of the number of low-brightness pixels LP in the mammary gland region MR after the correction.

In addition, in a case where a brightness value of the target breast ultrasound image TBUI is adjusted by the brightness value adjustment unit 20, the GTC calculation unit 38 may calculate a GTC indicating the ratio of the number of low-brightness pixels LP in the mammary gland region MR of the target breast ultrasound image TBUI in which the brightness value is adjusted.

In a case where there are a plurality of target breast ultrasound images TBUI, the GTC calculation unit 38 calculates a GTC for each of the target breast ultrasound images TBUI. A representative GTC is calculated based on a plurality of calculated GTCs. In the present embodiment, the GTC calculation unit 38 calculates an average GTC, which is an average value of the plurality of GTCs, as the representative GTC.

In a case where the threshold value specifying unit 36 specifies a maximum brightness threshold value and a minimum brightness threshold value in addition to the standard brightness threshold value, the GTC calculation unit 38 may calculate a maximum GTC which is a GTC based on the maximum brightness threshold value and a minimum GTC which is a GTC based on the minimum brightness threshold value. That is, the GTC calculation unit 38 calculates, as the maximum GTC, a parameter indicating a ratio of the number of low-brightness pixels LP in the mammary gland region MR in a case where a pixel group constituting the mammary gland region MR is divided into the high-brightness pixels HP of which the brightness value is equal to or more than the maximum brightness threshold value and the low-brightness pixels LP of which the brightness value is less than the maximum brightness threshold value. In addition, the GTC calculation unit 38 calculates, as the minimum GTC, a parameter indicating a ratio of the number of low-brightness pixels LP in the mammary gland region MR in a case where the pixel group constituting the mammary gland region MR is divided into the high-brightness pixels HP of which the brightness value is equal to or more than the minimum brightness threshold value and the low-brightness pixels LP of which the brightness value is less than the minimum brightness threshold value.

In a case where there are the plurality of target breast ultrasound images TBUI, the GTC calculation unit 38 can set a representative value (for example, an average value) of a plurality of maximum GTCs for each target breast ultrasound image TBUI as a representative maximum GTC. In addition, a representative value (for example, an average value) of a plurality of minimum GTCs for each target breast ultrasound image TBUI can be used as a representative minimum GTC.

The display controller 22 as a provision processing unit provides a GTC calculated by the GTC calculation unit 38 to a user by displaying the GTC on the display 24. FIG. 7 is a diagram illustrating an example of a GTC display screen. The display controller 22 displays the GTC calculated by the GTC calculation unit 38 on the display 24 as a numerical value P. In addition, the display controller 22 may display the target breast ultrasound image TBUI and the binarized image BI on the display 24, in addition to the numerical value P of the GTC. In a case where there are the plurality of target breast ultrasound images TBUI, the display controller 22 displays the representative GTC on the GTC display screen.

The user may be able to correct the mammary gland region MR and the fat region FR on the GTC display screen. For example, the user can input a correction instruction of the mammary gland region MR to the ultrasound diagnostic apparatus 10, by using the cursor Cu or the like on the GTC display screen. On the GTC display screen, the user may be able to designate the removal region ER. The mammary gland region specifying unit 32 corrects the mammary gland region MR in response to the correction instruction from the user. In addition, the user can input a correction instruction of the fat region FR to the ultrasound diagnostic apparatus 10, by using the cursor Cu or the like on the GTC display screen. The fat region specifying unit 34 corrects the fat region FR in response to the correction instruction from the user.

In a case where the mammary gland region MR or the fat region FR is corrected on the GTC display screen, the GTC may be changed. Therefore, in a case where the mammary gland region MR or the fat region FR is corrected on the GTC display screen, the threshold value specifying unit 36 specifies a brightness threshold value again based on the corrected fat region FR, and the GTC calculation unit 38 calculates a GTC again based on the mammary gland region MR after the correction and the re-specified brightness threshold value. The display controller 22 displays the re-calculated GTC as the numerical value P on the GTC display screen. In this manner, by correcting the mammary gland region MR or the fat region FR on the GTC display screen, the user can easily grasp the change of the GTC by the correction.

In addition, the user may be able to adjust a brightness value of each pixel constituting the target breast ultrasound image TBUI on the GTC display screen. In the present embodiment, the display controller 22 displays a brightness adjustment controller C for adjusting the brightness value of each pixel constituting the target breast ultrasound image TBUI on the GTC display screen. The user can input an instruction to adjust the brightness value of each pixel constituting the target breast ultrasound image TBUI to the ultrasound diagnostic apparatus 10, by operating the brightness adjustment controller C with the cursor Cu. The brightness value adjustment unit 20 adjusts the brightness value of each pixel constituting the target breast ultrasound image TBUI, in response to the adjustment instruction from the user.

In a case where the brightness value of each pixel constituting the target breast ultrasound image TBUI is adjusted on the GTC display screen, the GTC may be changed. Therefore, in a case where the brightness value of each pixel constituting the target breast ultrasound image TBUI is adjusted on the GTC display screen, the threshold value specifying unit 36 specifics a brightness threshold value again based on the adjusted brightness value of each pixel constituting the fat region FR of the target breast ultrasound image TBUI, and the GTC calculation unit 38 calculates a GTC indicating a ratio of the number of low-brightness pixels LP in the mammary gland region MR of the target breast ultrasound image TBUI of which the brightness value is adjusted. The display controller 22 displays the re-calculated GTC as the numerical value P on the GTC display screen. In this manner, by adjusting the brightness value of each pixel constituting the target breast ultrasound image TBUI on the GTC display screen, the user can easily grasp the change of the GTC by the adjustment of the brightness value.

Further, the display controller 22 may display a histogram H of the brightness value of each pixel constituting the mammary gland region MR on the GTC display screen. Further, in the histogram H, a brightness threshold value specified by the threshold value specifying unit 36 may be indicated. In the example of FIG. 7, a frequency of pixels having a brightness less than the brightness threshold value (that is, the low-brightness pixel LP) is illustrated by hatching, and a frequency of pixels having a brightness more than the brightness threshold value (that is, the high-brightness pixel HP) is illustrated in white. As a result, the user can graphically and intuitively grasp a ratio between the low-brightness pixel LP and the high-brightness pixel HP for each pixel constituting the mammary gland region MR.

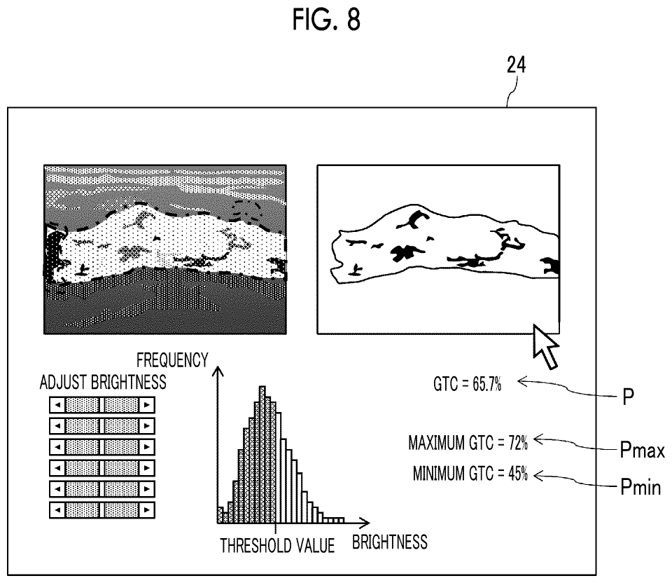

In a case where the GTC calculation unit 38 calculates a maximum GTC and a minimum GTC, the display controller 22 may provide the maximum GTC and the minimum GTC to a user. For example, as illustrated in FIG. 8, the display controller 22 may display a numerical value Pmax of the maximum GTC and a numerical value Pmin of the minimum GTC on the GTC display screen. In a case where there are the plurality of target breast ultrasound images TBUI, the display controller 22 may display a numerical value of the representative maximum GTC and a numerical value of the representative minimum GTC on the GTC display screen.

In addition, the display controller 22 may provide a difference between the maximum GTC and the minimum GTC (or a difference between the representative maximum GTC and the representative minimum GTC) to the user. For example, the display controller 22 may display a numerical value indicating the difference between the maximum GTC and the minimum GTC on the GTC display screen, instead of or in addition to the numerical value Pmax of the maximum GTC and the numerical value Pmin of the minimum GTC.

Further, the display controller 22 may notify the user in a case where the difference between the maximum GTC and the minimum GTC (or the difference between the representative maximum GTC and the representative minimum GTC) is equal to or more than a predetermined GTC threshold value. For example, in a case where the difference between the maximum GTC and the minimum GTC is equal to or more than the GTC threshold value, the display controller 22 may display a predetermined message on the GTC display screen.

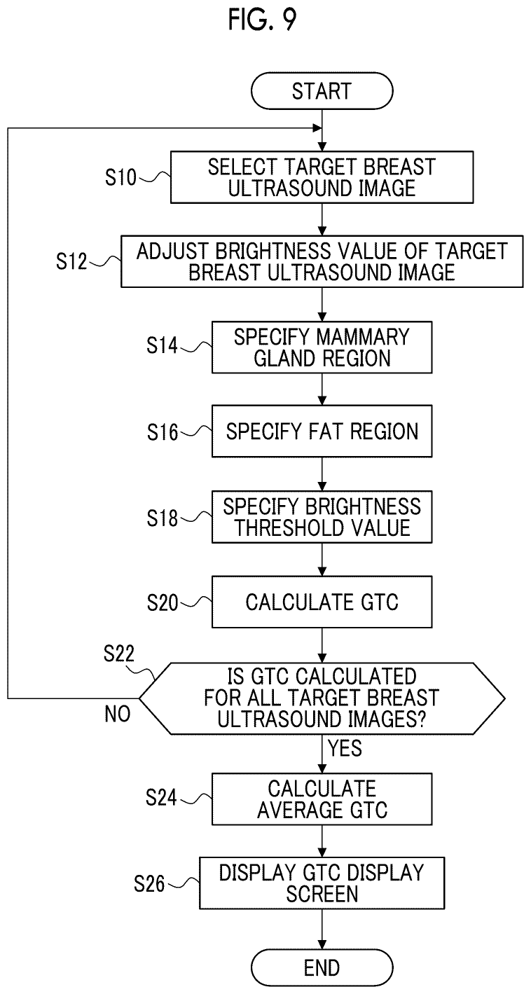

The outline of the configuration of the ultrasound diagnostic apparatus 10 according to the present embodiment is as described above. Hereinafter, a flow of the process of the ultrasound diagnostic apparatus 10 will be described with reference to a flowchart illustrated in FIG. 9.

In step S10, as a processing target, one target breast ultrasound image TBUI is selected from the plurality of target breast ultrasound images TBUI, which are B-mode images, formed by the image forming unit 18.

In step S12, the brightness value adjustment unit 20 adjusts a brightness value of each pixel constituting the target breast ultrasound image TBUI selected in step S10.

In step S14, the mammary gland region specifying unit 32 specifies the mammary gland region MR in the target breast ultrasound image TBUI.

In step S16, the fat region FR is specified in the target breast ultrasound image TBUI.

In step S18, the threshold value specifying unit 36 specifies a brightness threshold value, based on a brightness value of each pixel constituting the fat region FR specified by the fat region specifying unit 34.

In step S20, the GTC calculation unit 38 divides a pixel group constituting the mammary gland region MR specified in step S14 into the high-brightness pixel HP of which a brightness value is equal to or more than the brightness threshold value specified in step S18 and the low-brightness pixel LP of which a brightness value is less than the brightness threshold value. The GTC calculation unit 38 calculates a GTC that is a parameter indicating a ratio of the number of low-brightness pixels LP in the mammary gland region MR.

In step S22, the GTC calculation unit 38 determines whether or not the GTC is calculated for all the target breast ultrasound images TBUI scheduled to be processed. In a case where the target breast ultrasound image TBUI of which a GTC is not calculated remains, the process returns to step S10, and the process (calculating of the GTC) is performed on the other target breast ultrasound images TBUI in and after step S10. In a case where the GTC is calculated for all the target breast ultrasound images TBUI, the process proceeds to step S24.

In step S24, the GTC calculation unit 38 calculates an average GTC, which is a representative GTC of a plurality of GTCs calculated for each target breast ultrasound image TBUI.

In step S26, the display controller 22 displays a GTC display screen including the average GTC value calculated in step S24 on the display 24.

Although the ultrasound image analysis apparatus according to the present disclosure is described above, the ultrasound image analysis apparatus according to the present disclosure is not limited to the embodiment described above, and various modifications can be made without departing from the spirit of the present disclosure.

For example, in the embodiment described above, the ultrasound image analysis apparatus is the ultrasound diagnostic apparatus 10, but the ultrasound image analysis apparatus is not limited to the ultrasound diagnostic apparatus 10. For example, the ultrasound image analysis apparatus may be a personal computer (PC), a server having a processor, a memory, a communication interface, an input interface, and a display, or the like. In this case, the processor of the PC or the server as the ultrasound image analysis apparatus exerts functions as the brightness value adjustment unit 20, the display controller 22, the mammary gland region specifying unit 32, the fat region specifying unit 34, the threshold value specifying unit 36, and the GTC calculation unit 38, acquires the target breast ultrasound image TBUI from the ultrasound diagnostic apparatus or the like, and performs the GTC calculation process for the acquired target breast ultrasound image TBUI.

What is claimed is:

1. An ultrasound image analysis apparatus comprising:
a mammary gland region specifying unit that specifies a mammary gland region in a target breast ultrasound image, which is a processing target, formed by transmitting and receiving an ultrasound wave to and from a breast;
a fat region specifying unit that specifies a fat region in a breast ultrasound image formed by transmitting and receiving an ultrasound wave to and from a breast;
a threshold value specifying unit that specifies a brightness threshold value based on a brightness value of each pixel constituting the fat region;
a GTC calculation unit that divides a pixel group constituting the mammary gland region into a high-brightness pixel having a brightness value equal to or more than the brightness threshold value and a low-brightness pixel having a brightness value less than the brightness threshold value, and calculates a GTC indicating a ratio of the number of low-brightness pixels in the mammary gland region; and
a provision processing unit that provides the GTC to a user.

2. The ultrasound image analysis apparatus according to claim 1,
wherein the target breast ultrasound image and the specified mammary gland region are presented to the user,
the mammary gland region specifying unit corrects the mammary gland region in response to an instruction from the user, and
the GTC calculation unit calculates a GTC indicating a ratio of the number of low-brightness pixels in the mammary gland region after the correction.

3. The ultrasound image analysis apparatus according to claim 1,
wherein the breast ultrasound image is the target breast ultrasound image,
the ultrasound image analysis apparatus further comprises a brightness value adjustment unit that adjusts a brightness value of each pixel constituting the target breast ultrasound image,
the threshold value specifying unit specifies the brightness threshold value based on a brightness value after the adjustment of each pixel constituting the fat region of the target breast ultrasound image, and
the GTC calculation unit calculates a GTC indicating a ratio of the number of low-brightness pixels in the mammary gland region of the target breast ultrasound image of which the brightness value is adjusted.

4. The ultrasound image analysis apparatus according to claim 1,
wherein the target breast ultrasound image and the specified fat region are presented to the user,
the fat region specifying unit corrects the fat region in response to an instruction from the user, and
the threshold value specifying unit specifies the brightness threshold value based on a brightness value of each pixel constituting the fat region after the correction.

5. The ultrasound image analysis apparatus according to claim 1,
wherein the provision processing unit notifies the user in a case where a variation in the brightness value of each pixel constituting the fat region is equal to or more than a variation threshold value.

6. The ultrasound image analysis apparatus according to claim 1,
wherein the threshold value specifying unit specifies a maximum brightness threshold value in a brightness threshold value width that is an acceptable width of the brightness threshold value and a minimum brightness threshold value in the brightness threshold value width, based on a variation in the brightness value of each pixel constituting the fat region,
the GTC calculation unit calculates a maximum GTC that is the GTC based on the maximum brightness threshold value and a minimum GTC that is the GTC based on the minimum brightness threshold value, and
the provision processing unit provides at least one of the maximum GTC and the minimum GTC or a difference between the maximum GTC and the minimum GTC to the user, or notifies the user in a case where the difference between the maximum GTC and the minimum GTC is equal to or more than a GTC threshold value.

7. A non-transitory computer-readable storage medium storing an ultrasound image analysis program causing a computer to function as:
a mammary gland region specifying unit that specifies a mammary gland region in a target breast ultrasound image, which is a processing target, formed by transmitting and receiving an ultrasound wave to and from a breast;
a fat region specifying unit that specifies a fat region in a breast ultrasound image formed by transmitting and receiving an ultrasound wave to and from a breast;
a threshold value specifying unit that specifies a brightness threshold value based on a brightness value of each pixel constituting the fat region;
a GTC calculation unit that divides a pixel group constituting the mammary gland region into a high-brightness pixel having a brightness value equal to or more than the brightness threshold value and a low-brightness pixel having a brightness value less than the brightness threshold value, and calculates a GTC indicating a ratio of the number of low-brightness pixels in the mammary gland region; and
a provision processing unit that provides the GTC to a user.
